(19) United States
(12) Plant Patent Application Publication
(10) Pub. No.: US 2002/0073471 P1
(43) Pub. Date: Jun. 13, 2002

(54) Variety of geranium plant named 'Pensyb'

(76) Inventor: Andrea Michalik, Dresden (DE)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 09/733,648

(22) Filed: Dec. 8, 2000

(51) Int. Cl. A01H 5/00

(52) U.S. Cl. PLT/332

(57) ABSTRACT

A new and distinct zonal geranium plant with red colored flowers above medium green foliage. The plant is suitable for planting in small pots.

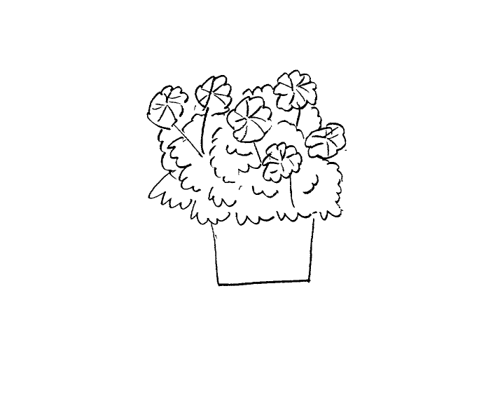

### BACKGROUND OF THE INVENTION

The present invention comprises a new and distinct cultivar of Pelargonium peltatum known by the varietal name ‘Pensyb’. The new variety was discovered in 1995 in a selective breeding program in Dresden, Germany. The new variety is the result of cross breeding of a group of about 50 seedlings having double dark red colored flowers via embroy rescue. The new variety was first asexually reproduced by cuttings in Dresden, Germany in 1995. The new variety has been trial and field tested and has been found to retain its distinctive characteristics and remain true to type through successive propagations.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Dresden, Germany. The color readings were taken outdoors in shadow during summer. Color references are primarily to The R.H.S. Colour Chart of The Royal Horticultural Society of London.

Plant:

Classification.—Botanical: Pelargonium peltatum. Commercial: Zonal geranium.

Form.—Hanging.

Height from media surface to top of foliage.—25-40 cm.

Height from media surface to top of umbel.—25-40 cm.

Growth.—High degree of branching.

Strength.—Does not require artificial support.

Response time.—4 weeks.

Leaves.—Size:: 4-5 cm long; 5-6 cm wide. Shape: Overall: Ivy-shaped. Base: Overlapped. Apex: Pointed. Margin: Emarginated. Texture: Pubescent. Color: Upper surface: Green Group 136B. Lower surface: Green Group 138B. Ribs and veins: Venation: Net-like. Color: Brighter than leaf surface.

Petioles.—Color: Medium green. Length: 2-3 cm.

Stem/branches.—Color: Medium green. Internode length: 1-2 cm. Lateral branch length: 10-15 cm.

The bud:

Shape when just showing color.—Overall: Spherical. Individual bud: Elliptical.

Color.—Green Group 138A.

Size when just showing color.—Umbel: 5-6 cm across. Individual bud: 0.7 long; 0.7-1 cm wide.

Inflorescence:

Blooming habit.—Double.

Borne.—Flowers on umbel; umbel on pedicel; pedicel on peduncle.

Umbel.—6-8 cm across; 3-4 cm deep.

Flowers.—Number of flowers: 7-9. Form: Round. Number of petals: 17-20. Size: 3-3.5 cm across; 1-1.5 cm deep. Petals: Size: 1.5 cm long; 1 cm long. Color: Upper surface: Red Group 53B. Lower surface: Red Group 43C. Shape: Overall: Round-elliptical. Apex: Round. Base: Narrow. Sepals: Color: Upper surface: Green Group 143B. Lower surface: Green Group 143B. Shape: Overall: Elliptical. Apex: Pointed Base: Blunt. Size: 1.3 cm×0.3 cm. Texture and appearance: Velvety. Tonality from a distance: Rose-like impression of the flower buds.

Petaloids.—Quantity: 12-15. Shape: Round; irregular. Color: Red Group 53B.

Pedicel.—Length: 1.0-1.5 cm. Color: Green Group 1 37C.

Peduncle.—Length: 7-9 cm. Color: Green Group 141 C.

Disease/pest resistance.—No unusual susceptibility to diseases or pests has been noted to date.

Lasting quality.—Long-lasting; early flowering.

Natural flowering season.—Summer, frost free period.

Reproductive organs:

Stamens.—Anthers: 3 mm long. Filaments: Length: 0.5 cm maximum. Color: Pink. Pollen: Yellow; scant.

Pistils.—Number: Five. Length: 0.3-0.4 cm. Stigma: Pink. Style: 0.8 cm long. Color: Greenish.

Ovaries.—Superior; some pubescence; greenish; 0.4 cm long.

Fruit.—Not present.

### GENERAL CHARACTERISTICS

1. Very compact.

2. Very early flowering.

3. Rose bud type.

4. Ideal for small pots.

5. Stable flower color under sun.

I claim:

1. A new and distinct variety of geranium plant substantially as shown and described.
